(19) United States
(12) Patent Application Publication
(10) Pub. No.: US 2006/0233846 A1
(43) Pub. Date: Oct. 19, 2006

(54) Microdispersion and method of producing same

(75) Inventors: Steve Leigh, Muttenz (CH); Mathew Louis Steven Leigh, Basel (CH); Peter Van Hoogevest, Bubendorf (CH); Elsa Kung, Basel (CH)

Correspondence Address: BUCHANAN, INGERSOLL & ROONEY PC, POST OFFICE BOX 1404, ALEXANDRIA, VA, 22313-1404, US

(73) Assignee: PHARES PHARMACEUTICAL RESEARCH N.V., Curracao (NL)

(21) Appl. No.: 10/532,309

(22) PCT Filed: Oct. 28, 2003
(86) PCT No.: PCT/EP03/11936

§ 371 Date: Mar. 14, 2006

(30) Foreign Application Priority Data
Oct. 28, 2002 (EP) 02 257 455.2

(51) Int. Cl. A61K 8/02

(52) U.S. Cl. 424/401

(57) ABSTRACT

The invention describes homogeneous microdispersions comprising at least one hydrogenated or partially hydrogenated membrane lipid with or without enzyme hydrolysis, dispersed in substantially non aqueous, non volatile hydrophilic medium with boiling point above 40° C. More preferably the compositions comprise a mixture of hydrogenated monoacyl and hydrogenated diacyl-lipids and at least one oil. The phospholipid mixture is obtained by controlled enzyme hydrolysis of lecithin or a specific phospholipid, followed by hydrogenation. The compositions have improved rheology, physical and chemical properties, functionality and industrial applicability. The microdispersions are used as such in all types of applications and as functional components with active compounds in products, particularly for improving skin function and facilitating skin repair due to UV damage and aging, in cosmetics and other topical products.

### FIELD OF THE INVENTION

This invention concerns membrane lipid compositions and a method of preparing microdispersions comprising membrane lipids with either saturated or partially saturated diacyl or monoacyl chains in a substantially non aqueous medium.

### BACKGROUND TO THE INVENTION

Phospholipids are the most abundant membrane lipid found in living cells. Diacyl membrane lipids have twin fatty acid hydrocarbon chains attached to a glycerol backbone in the 1 and 2 position and a polar head group in position 3. However, they can also have single, monoacyl chains. The hydrocarbon chains attached to natural phospholipids are mostly diacyl comprising 14 to 24 carbon fatty acids. The monoacyl components are classed as breakdown products totalling less than 3%. The physical state of phospholipids is defined by the phase transition temperature (Tc). Below the phase transition temperature, the lipid molecules are arranged in a solid, gel state. Above the Tc, the lipid molecules assume a liquid crystalline state. The hydrocarbon chains of most natural phospholipids are unsaturated and may contain between one to six double bonds depending on the type of fatty acid and the source, e.g. marine, animal, or plant. The Tc of natural phospholipids comprising unsaturated fatty acids is in the region of −10° C. to −20° C.

Phospholipids and to a lesser extent glycolipids and ceramides have ubiquitous and multi-functional applications for oral, topical and industrial uses. Lecithin is a crude mixture of different types of natural phospholipids used in formulations as excipient to improve stability and performance. Phospholipids are used with glycolipids and ceramides as biologically active compounds to improve skin functions and as natural moisturisers with emollient and skin ‘regeneration’ properties. Egg phospholipids are used most frequently in the pharmaceutical industry as emulsifiers in parenteral nutrition and other intravenous injections. There is also wide interest in delivery systems using liposomes comprising phospholipids as the main component for entrapment and targeted delivery of biologically active compounds.

Notwithstanding the extensive use of phospholipids in all types of application, a practical problem concerns oxidative degradation of unsaturated bonds in the fatty acid chains. This seriously limits the use of natural phospholipids from soya and egg in products where sensoric and cosmetic properties are important. Hydrogenation overcomes the problem but it also decreases dispersibility in both water and oil. Therefore from practical considerations it is not easy to incorporate hydrogenated lipids which may be chemically more stable into products and processes. There is thus an industrial need for improved, saturated or partially saturated membrane lipid compositions comprising either single or twin hydrocarbon chains which are easy to incorporate into products and processes.

### PRIOR ART

The prior art on phospholipid compositions generally describes methods for preparing vesicular structures for entrapment and drug delivery. The disclosures are aimed at obtaining maximum entrapment and improved delivery of compounds by means of closely defined vesicles dispersed in water which must remain intact with minimum leakage during storage. These features are typically disclosed e.g. in EP-A-0 158 441 and U.S. Pat. No. 5,169,637. For background on liposomes in drug delivery, reference is made to Drug Development and Industrial Pharmacy, 15(10), 1523-1554 (1989).

The phase behaviour of microemulsion systems containing lecithin and lysolecithin as surfactants is described in International Journal of Pharmaceutics 143 (1996) 67-73. The phase diagrams studied were obtained from compositions comprising unsaturated phospholipids in a volatile co solvent such as ethanol, butanol and propanol, a lipophilic phase, and water. They do not include anhydrous systems.

WO 98/58629 describes compositions comprising combinations of monoacyl and diacyl membrane lipids. WO 00/61113 further describes homogeneous formulations for forming dispersed compositions which may be microemulsions comprising membrane lipids and enzyme modified lipids for solubilising compounds and improving bioavailability. The present invention is a further specific development and describes substantially non aqueous microdispersed, colloidal compositions which term includes microemulsions, comprising hydrogenated membrane lipids dissolved in nano-size oil globules and dispersed in a substantially non aqueous hydrophilic medium. Furthermore, where the invention includes a microemulsion composition, intensive work energy is required to form nano-size oil globules for carrying the hydrogenated membrane lipids.

EP-A-0 953 339 describes a composition for cosmetic use which comprises a lysophospholipid mixture, wherein 30 mol % or more of fatty acids bonded to said lysophospholipid mixture are monoenoic fatty acids derived from safflower oils. The compositions are claimed to have superior organoleptic and stability properties compared to hydrogenated lyso lecithin which comprise saturated fatty acids that contain less than 30% oleic acid, a monoenoic acid.

JP 3139246 describes compositions comprising 90%-99% by weight of lyso phospholipids and 1% to 10% by weight of a medium chain triglyceride to improve water dispersibility.

### SUMMARY OF THE INVENTION

The present invention is in the area of ‘non aqueous hydrophilic microdispersions’ and ‘hydrogenated membrane lipid compositions’.

The invention describes homogeneous microdispersions comprising at least one hydrogenated, partially hydrogenated, saturated or partially saturated membrane lipid, with or without enzyme hydrolysis, dispersed in substantially non aqueous, non volatile hydrophilic medium with boiling point above 40° C. Optionally the compositions may comprise biologically active compounds, excipients and preservatives such as antioxidants, anti-microbials, buffering agents in a non aqueous system.

More preferably the compositions comprise a mixture of hydrogenated monoacyl and hydrogenated diacyl lipids and an oil. The mixture of diacyl and monoacyl lipid phospholipids is obtained by controlled enzyme hydrolysis of lecithin or a specified phospholipid, followed by hydrogenation. The compositions have improved rheology, physical and chemical properties, functionality and industrial applicability. The microdispersions are used as such in all types of applications and as functional components with active compounds in products, particularly for improving skin function in cosmetics and other topical products. The compositions have the potential to stimulate the Basement Membrane (BM) layer located at the dermal-epidermal junction. The BM is involved in the repair and regeneration process due to aging or UV exposure and may provide anchoring fibrils involving collagen to keep the skin firm. More generally, they may be used as excipients with other components used in food, pharmaceuticals, aqua culture, agriculture and horticulture, etc. The invention provides a more convenient means to incorporate hydrogenated, and saturated membrane lipids in a molecularly dispersed state in all types of processes, applications and products.

### DETAILED DESCRIPTION OF THE INVENTION

The compositions in this invention are microdispersions comprising hydrogenated, or saturated and partially saturated membrane lipid particles and preferably at least one oil dispersed in substantially non aqueous hydrophilic medium. The oil may be a fixed or a volatile oil.

The definition of ‘microdispersion’ includes oil in water (o/w) type non aqueous microemulsions or suspended oil droplets below 1000 nm average diameter using laser diffraction measurements.

The term ‘hydrogenated’ includes saturated and partially saturated membrane lipids with acyl chains thar comprise less than about 30 mol % of unsaturated fatty acids. The saturated fatty acids may be present naturally or they may be prepared by hydrogenation using a catalyst. Lipid further refers to membrane lipids with one or two hydrocarbon chains and include all types of phospholipids, glycolipids and ceramides.

Accordingly the present invention describes a homogeneous microdispersion comprising i) a dispersed phase comprising at least one hydrogenated membrane lipid with or without enzyme hydrolysis, and preferably one or more oils, ii) a substantially non aqueous hydrophilic phase which is substantially free of volatile organic solvents, iii) optionally one or more biologically active compounds, excipients, preservatives, etc.

The invention further describes a method which involves preparing said micro dispersions comprising hydrogenated lipids by applying intensive energy at elevated temperatures to substantially non aqueous medium and an oil to obtain dispersed lipid particles that are below about 5000 nm, preferably below 1000 nm z average diameter, most preferably between about 10 nm to 500 nm. Substantially non aqueous medium enables larger amounts of the saturated or partially membrane lipid to be dispersed as lipid aggregates in a fluid medium without making the composition too viscous.

Dispersed Phase

The dispersed phase in the microdispersions may be from 0.1% to as much as 50% by weight of the composition. In one embodiment, it may consist of only one hydrogenated membrane lipid such as a diacyl phospholipid on its own dispersed in a substantially non aqueous hydrophilic medium. In a preferred embodiment, the dispersed phase comprises a combination of hydrogenated diacyl and monoacyl phospholipids obtained by enzyme hydrolysis. In particularly preferred embodiments, the dispersed phase comprises mixtures of hydrogenated monoacyl and diacyl lipids and an oil, in oil-in-water type non aqueous microemulsions.

The micro dispersions may comprise between 0.01% to 40%, preferably 1% to 20% by weight of either hydrogenated phospholipids (including lipids with at least 70% of naturally saturated fatty acids) on their own or enzyme modified and hydrogenated phospholipids.

Typically, crude lecithin from soya is a mixture with about 40 wt % non polar fatty acid glycerides and 60 wt % polar lipids of which 80% to 85% are phospholipids and the rest glycolipids and phosphorus free polar lipids. Therefore phospholipids account for about 50% by weight of the total mixture with phosphatidyl choline (PC) as the major component at approximately 15% and phosphatidyl ethanolamine (PE) at about 10%. The rest are phosphatidyl inositol (PI), phosphatidic acid (PA), phosphatidylserine (PS), etc. The fatty acid chains of plant derived phospholipids are mostly unsaturated with 16 to 18 carbon atoms and one to three double bonds.

The PC content of the hydrogenated lipid used in this invention may range from about 15% to 95% by weight. Hydrogenation may be carried out on crude lecithin compositions as described above comprising about 15% PC. However the deoiled material with approximately 20% to 25% PC content is preferred. More preferably a de oiled and fractionated material with 25% to 50% PC is used for hydrogenation. It is also possible to hydrogenate purer fractions comprising more than 50% PC to obtain up to 95% hydrogenated PC. A catalyst such as palladium on carbon black is normally employed for hydrogenation.

The lipids covered by this invention include membrane lipids where the acyl chains comprise at least 70% of naturally saturated, or semi-synthetically hydrogenated fatty acids with 10 to 36, preferably 14 to 24 carbon atoms.

Enzyme hydrolysis is carried out using phospholipase A1 or A2 to cleave off one of the two fatty acid chains from the diacyl lipid prior to hydrogenation. The enzyme modified material used in this invention may contain between 5% to 90% by weight of mono acyl components in the total mixture. This figure, referred to as the conversion rate or degree of hydrolysis is based on the conversion rate of the major component phosphatidylcholine. Preferably the conversion rate is between 10% to 65%. More preferably it is between 15% to 35%. The desired level of monoacyl PC in the final composition is usually obtained by back blending a hydrolysed lipid mixture with appropriate amounts of hydrogenated diacyl PC. As a rule, hydrogenation is always carried out on either the diacyl phospholipid mixtures or monoacyl and diacyl lipid mixtures from enzyme treatment, after fractionation and purification.

Oil Component

The invention further allows for the dispersed phase to include one or more oils in a non aqueous microemulsion. In this particular embodiment, the oil provides a lipophilic domain for the hydrocarbon chains in the hydrogenated lipids. The oil may comprise from 0% to 40% by weight of the microdispersion. Preferably it comprises 5% to 30% by weight, most preferably 10% to 20% by weight of the total components.

The oil may be any fixed or volatileoil, a hydrocarbon, a silicon oil, or combinations thereof. It may be a natural vegetable oil or synthetic medium chain mono, di or tri glycerides or a mixture of all three glycerides containing 12 to 20 carbon atoms. For external and topical applications synthetic and semi synthetic fatty acid ethers and esters such as isopropyl myristate and isopropyl palmitate and long chain alcohols such as oleyl alcohol are suitable alternatives. Particularly suitable oils are the alpha tocopherols, Vit D oily solutions and wheat germ oil. It should be clearly understood that there is no restriction on the type of oil that may be used. The oil is employed to i) provide a lipophilic domain to associate with the hydrocarbon tails of the hydrogenated lipids and thereby render the lipids more dispersible and less viscous with better flow properties, ii) confer additional emolliency and other physiological benefits that may be desired. Therefore any suitable oil on its own or blends that can provide a useful function may be used.

Hydrophilic Medium

The hydrophilic phase comprises from about 10% to 90% by weight of the composition. Preferably from 20% to 50% by weight. The hydrophilic phase forms the continuous medium to facilitate dispersion and miscibility in water. Preferably it comprises polar liquids with more than one hydroxyl group that is not a good solvent for the lipid. Glycerol is a preferred polyhydroxy hydrophilic medium. Minor amounts of water may be present as long as the continuous medium is substantially non aqueous. For practical purposes it is difficult to remove water entirely from hydrophilic liquids such as glycerol and therefore commercial grades may contain up to about 10% or more water. The reasons for avoiding larger amounts of water are, i) compared to non aqueous polar liquids such as glycerol, the hydrophilic regions in fully hydrated bilayers in water are more expanded, increasing the viscosity of the composition and restricting the amount of membrane lipids that can be used, ii) water encourages microbial contamination and growth. Therefore, the amount of water in the microdispersion is best limited to about 20%, preferably 10% to 15% by weight and more preferably less than 5%. The exception is concentrated sugar solutions where the water is bound to the hydroxyl groups. Therefore, polyhydric alcohols such as concentrated sugar solutions below about 70%, preferably below 50% by weight of water are suitable. They may be hexose or pentose sugars such as sucrose, dextrose, mannitol, sorbitol or xylitol, etc. These concentrated sugar solutions may be used on their own or in combination with non aqueous hydrophilic liquids so that the overall water content of the lipid microdispersions is kept below about 20% to 30% by weight. Preferred non aqueous components that may be used include but are not limited to non volatile liquids e.g. propylene glycol, glycerol, butylene glycol, hexylene glycol, etc and mixtures thereof. The hydrophilic phase is non volatile and will have a boiling point above ambient, preferably above about 40° C.

The amount of the microdispersion used on its own or in a composition may range from 0.1% to 99% by weight. Typically they cover the range from 1% to 50% and preferably from 5% to 25% by weight of the total composition. The microdispersion is particularly suitable for incorporating into preparations for topical use such as a cream, ointment, spray, a gel or a transdermal system.

Method

At least one hydrogenated lipid and preferably one or more oil component is dispersed in a non aqueous hydrophilic medium using a homogeniser with high speed stirrer for approximately 4-5 minutes at a speed of 13500-87500 revolutions per minute—at temperatures above 30° C. depending on the proportions of the dispersed phase and the hydrophilic phase, to obtain a coarse primary emulsion. The primary composition is put through an Avestin Emulsiflex C5 micro-fluidiser maintained at an elevated temperatures to prepare compositions which disperse readily in water to give a clear dispersion. The number of cycles required depends upon the viscosity of the primary emulsion prior to homogenising. The specification range typically for the above micro fluidiser is given below. Higher pressures may be employed using other types of equipment. The essential requirement is to prepare microdispersions which in the nano size range mostly below 1000 nm z average diameter. Thus other equipment such as high pressure extrusion, high impact milling, high shear mixing, sonication and other homogenising equipment which disrupt the dispersed phase and reduce it to nano size lipid particles are all suitable.

Homogenising pressure: 5000-25000 psi

air/gas inlet pressure: 30-80 psi

### EXAMPLE 1

| | |
|---|---|
| Hydrogenated lecithin * | 10% w/w |
| Miglyol 810N | 10% w/w |
| Glycerol | 80% w/w |

* Comprises about 80% total phospholipids with about 23% diacyl phosphatidylcholine and less than 1% monoacyl PC as impurity. Over 70% of the phospholipids are saturated.

The hydrogenated lipid is dispersed in the oil and the glycerol to form a coarse primary o/w emulsion at elevated at an temperature between 50° C. to 60° C. This is processed to give a homogeneous microemulsion using an Avestin Emulsiflex micro-fluidiser provided with heating means to maintain the temperature above 50° C., to obtain nano oil droplets with a z average diameter below 200 nm.

This composition may be added to a cream.

### EXAMPLE 2

| | |
|---|---|
| Hydrogenated, enzyme modified phospholipid * | 15% w/w |
| Isopropyl palmitate | 15% w/w |
| Butylene glycol | 75% w/w |

* Comprises 60% of diacylphosphatidylcholine and about 20% of monoacyl phosphati-dylcholine. Over 90% of the phospholipids are saturated.

The microdispersion comprising hydrogenated and enzyme modified lecithin is prepared as in example 1. In this case the dispersion is processed in the micro-fluidiser at a temperature above 50° C. until the lipid particles are below 500 nm z average diameter. The microemulsion obtained was slightly less translucent and disperses readily in water or an oil with agitation. It is suitable for adding to a clear gel composition.

### EXAMPLE 3

| | | |
|---|---|---|
| Enzyme modified, hydrogenated phospholipid | 7.5% | w/w |
| Glycerol | 92.5% | w/w |

The lipid used in EXAMPLE 3 was heated in the glycerol at about 70° C. and the nano dispersion was prepared using an Ultra Turrax vortex mixer at intermediate speed. A translucent dispersion with good flow properties comprising lipid particles below 100 nm was obtained. The composition disperses easily in water.

### SUMMARY

The invention describes homogeneous microdispersions comprising at least one hydrogenated or partially hydrogenated membrane lipid with or without enzyme hydrolysis, dispersed in substantially non aqueous, non volatile hydrophilic medium with boiling point above 40° C. More preferably the compositions comprise a mixture of hydrogenated monoacyl and hydrogenated diacyl lipids and at least one oil. The phospholipid mixture is obtained by controlled enzyme hydrolysis of lecithin or a specific phospholipid, followed by hydrogenation. The compositions have improved rheology, physical and chemical properties, functionality and industrial applicability. The microdispersions are used as such in all types of applications and as functional components with active compounds in products, particularly for improving skin function and facilitating skin repair due to UV damage and aging, in cosmetics and other topical products.

1. A microdispersion comprising:
at least one hydrogenated or partially hydrogenated, saturated or partially saturated membrane lipid with or without enzyme hydrolysis dispersed homogeneously in a substantially non aqueous and non volatile hydrophilic medium.

2. Microdispersion according to claim 1, wherein the dispersed particles are below 1000 nm.

3. Microdispersion according to claim 1, wherein the dispersed particles include oil droplets comprising between 0 wt % to 40 wt % of at least one oil associated with at least one hydrogenated or partially hydrogenated membrane lipid with a particle size below 1000 nm z average diameter.

4. Microdispersion according to claim 1, wherein the dispersed phase comprises 0.1 wt % to 50 wt % of the total components.

5. Microdispersion according to claim 1, wherein the dispersed phase comprises between 0.01 wt % to 40 wt % of hydrogenated/saturated diacyl membrane lipids with at least 70 mol % of saturated fatty acids.

6. Microdispersion according to claim 1, wherein the dispersed phase comprises between 0.01 wt % to 40 wt % mixture of hydrogenated/saturated diacyl and monoacyl membrane lipids with at least 70% of saturated fatty adds.

7. Microdispersion according to claim 5, wherein the hydrogenated membrane lipids are enzyme modified and comprise between 5 wt % to 90 wt % of monoacyl phosphatidylcholine.

8. Microdispersion according to claim 1, wherein the non aqueous hydrophilic medium comprises between 10 wt % to 90 wt % of at least one non volatile liquid with boiling point above 40° C.

9. A method of preparing a microdispersion according to claim 1, which comprises a step that involves dispersing at least one hydrogenated membrane lipid with or without enzyme modification in a substantially non aqueous hydrophilic medium by mixing above ambient temperatures in order to obtain dispersed particles below 1000 nm z average diameter.

10. Microdispersion according to claim 1 for incorporation into a topical composition.

11. A composition including the microdispersion of claim 1.

12. Microdisperion of claim 1, comprising:
biologically active compounds, excipients and preservatives.

13. Microdispersions of claim 1, with enzyme hydrolysis.

14. Microdispersion according to claim 2, wherein the dispersed particles include oil droplets comprising between 0 wt % to 40 wt % of at least one oil associated with at least one hydrogenated or partially hydrogenated membrane lipid with a particle size below 1000 nm z average diameter.

15. Microdispersion according claim 14, wherein the dispersed phase comprises 0.1 wt % to 50 wt % of the total components.

16. Microdispersion according to claim 15, wherein the dispersed phase comprises between 0.01 wt % to 40 wt % of hydrogenated/saturated diacyl membrane lipids with at least 70 mol % of saturated fatty acids.

17. Microdispersion according claim 16, wherein the dispersed phase comprises between 0.01 wt % to 40 wt % mixture of hydrogenated/saturated diacyl and monoacyl membrane lipids with at least 70% of saturated fatty adds.

18. Microdispersion according to claim 17, wherein the hydrogenated membrane lipids are enzyme modified and comprise between 5 wt % to 90 wt % of monoacyl phosphatidylcholine.

19. Microdispersion according to claim 18, wherein the non aqueous hydrophilic medium comprises between 10 wt % to 90 wt % of at least one non volatile liquid with boiling point above 40° C.

20. A method of preparing a microdispersion according to claim 19, which comprises a step that involves dispersing at least one hydrogenated membrane lipid with or without enzyme modification in a substantially non aqueous hydrophilic medium by mixing above ambient temperatures in order to obtain dispersed particles below 1000 nm z average diameter.
